(19) United States
(12) Patent Application Publication
(10) Pub. No.: US 2021/0364178 A1
(43) Pub. Date: Nov. 25, 2021

(54) AIR AND SURFACE DISINFECTING SYSTEM

(71) Applicant: Medical Illumination International Inc., San Fernando, CA (US)

(72) Inventor: Alan Kiviat, Studio City, CA (US)

(21) Appl. No.: 17/327,314

(22) Filed: May 21, 2021

Related U.S. Application Data
(60) Provisional application No. 63/029,463, filed on May 23, 2020.
(60) Provisional application No. 63/035,657, filed on Jun. 5, 2020.

(51) Int. Cl. F24F 8/22; F24F 3/163

(52) U.S. Cl. CPC F24F 8/22; F24F 3/163

(57) ABSTRACT

Provided is a disinfecting fixture comprising: a) a body; b) a fan; c) a first irradiation source placed inside of the body, the irradiation source inside of the body configured to disinfect air forced into the body by the fan; d) a conduit for movement of air from outside of the body to inside of the body for irradiation with the first irradiation source, and movement of air outside of the body after irradiation with the first irradiation source; and e) a second irradiation configured to irradiate air and/or surfaces outside of the body; wherein air moves with a force generated by the fan from outside of the fixture to inside of the fixture for irradiation with the first irradiation source, and exits the fixture, and wherein air outside of the fixture and/or surfaces below the fixture are irradiated with the second irradiation source or sources. Provided is a method for disinfecting light comprising: a) creating air flow from outside to inside and then outside of a fixture with a fan or fans; b) irradiating the air moved inside of body; c) moving the irradiated air outside with the flow of the air; and d) irradiating air and/or surfaces outside of the fixture with a second irradiation source or sources placed outside of the body.

### CROSS-REFERENCE

The present application claims the benefit of U.S. Provisional patent application No. 63/029,463 filed on May 23, 2020, and U.S. provisional patent application No. 63/035,657, filed on Jun. 5, 2020, all of which are incorporated by reference here in their entirety.

Disclosed is an ultraviolet light, antimicrobial and antiviral, environmental air and surface disinfecting system and light fixture system.

### BACKGROUND

A UV-C system combines an ultraviolet germicidal irradiation (UVGI) chamber and air circulating fans. The system uses UV-C light and filtration to draw in and treat environmental air on one side and return air to the room through an exhaust on the other side, reducing microbial and fungal populations in treated air and reduces settling bacteria, viruses, and fungi from treated air. A problem with such systems is that there is a need for additional disinfection, which may not be possible with existing systems.

### DETAILED DESCRIPTION OF THE DRAWINGS

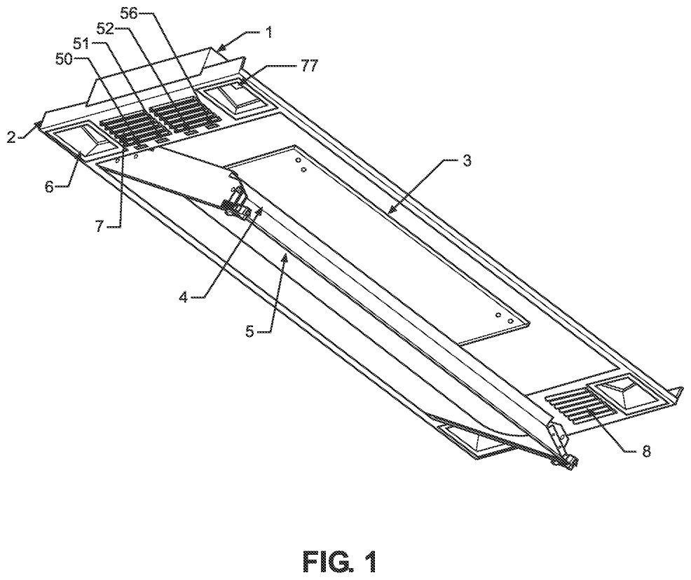

FIG. 1 shows a bottom view of an embodiment with the light panel open, for example for maintenance or lamp or filter replacement.

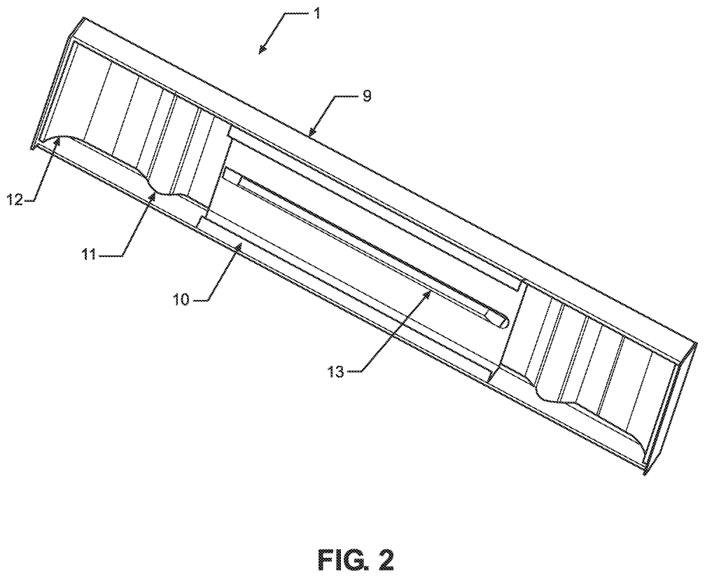

FIG. 2 shows top section of irradiation chamber.

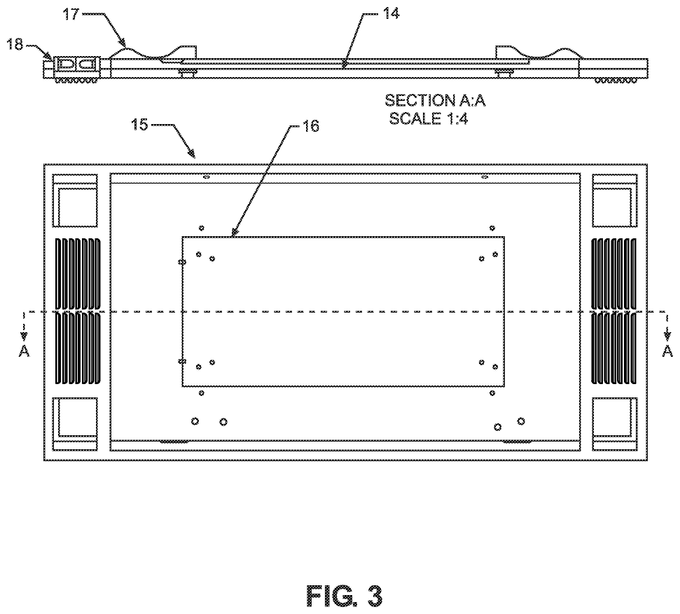

FIG. 3 illustrates a Main body assembly.

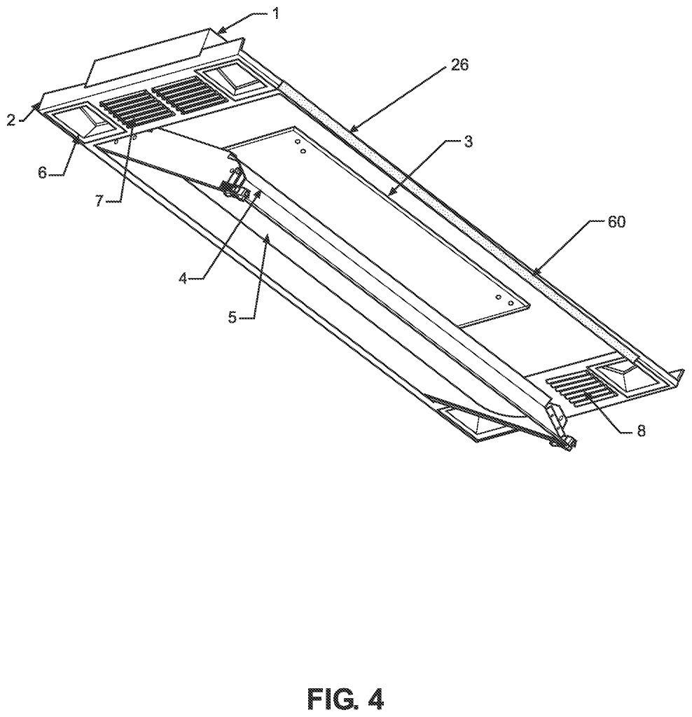

FIG. 4 illustrates the disinfecting system with an optional UV light board.

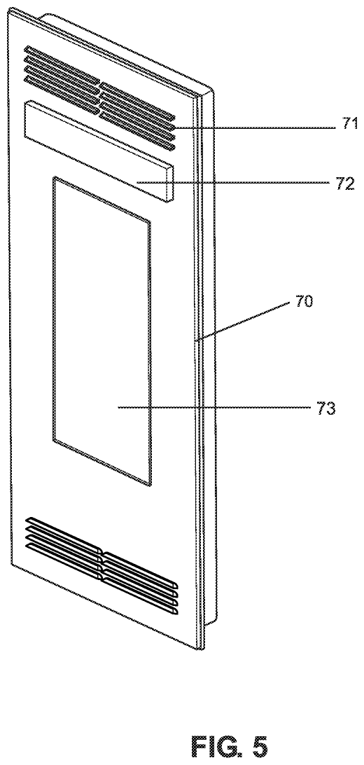

FIG. 5 illustrates a wall mounted unit.

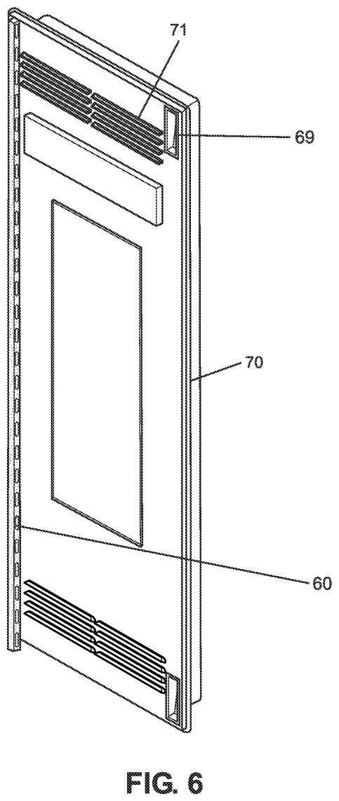

FIG. 6 illustrates a wall mounted unit.

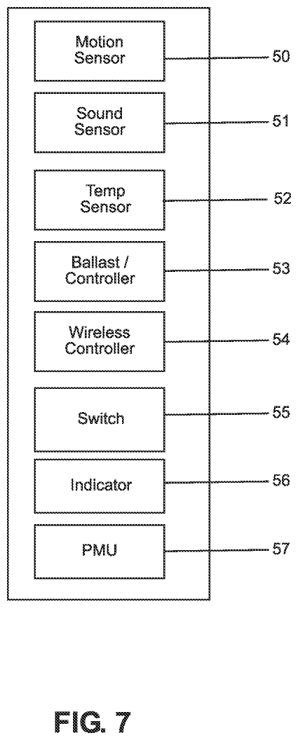

FIG. 7 illustrates various electronic components (plus filter) of the disinfecting system.

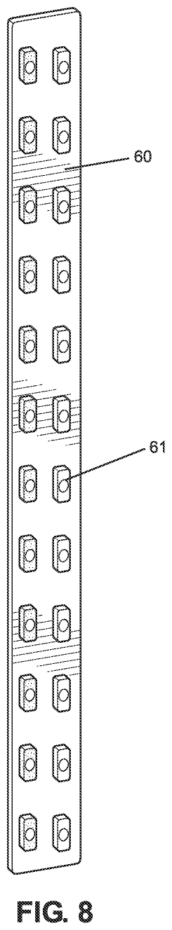

FIG. 8 illustrates an LED board with an LED source of light.

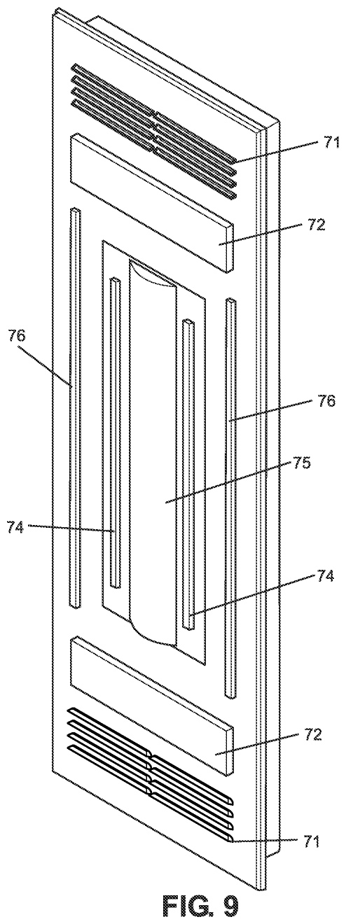

FIG. 9 illustrates placement of UV and or non-UV lights on outside of the body.

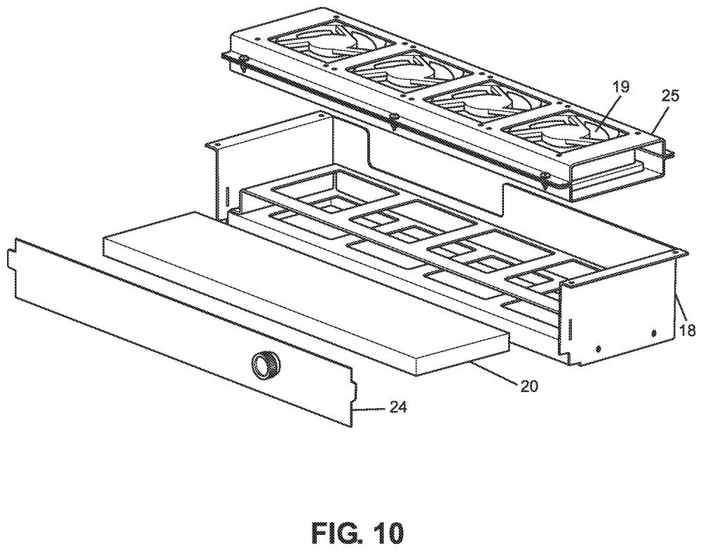

FIG. 10 illustrates the Fan Enclosure/Filter Holder.

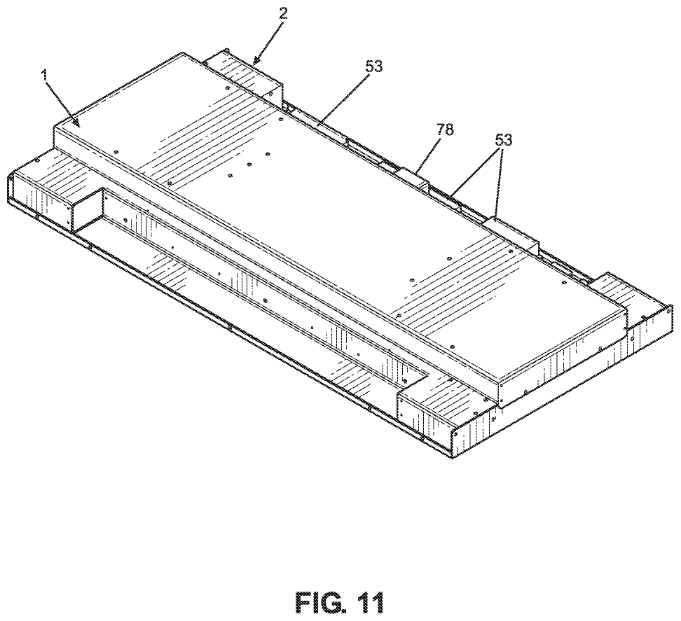

FIG. 11 illustrates a top view of the light panel closed.

### SUMMARY SECTION OF THE INVENTION

Provided in an embodiment is a disinfecting fixture configured to be attached to a ceiling, the fixture comprising: a) a body; b) fan or array of fans; c) a first irradiation source inside of the body, the irradiation source inside of the body configured to disinfect air forced into the body by the fan; d) a second irradiation source configured to irradiate (air or surface) outside of the body; e) a door attached to the body for accessing inside of the body; f) optionally one or more light sources to provide illumination. The door of any of the above embodiments can be pivotally attached on the long side of the body, which is typically rectangular in shape. In any of the above embodiments, the ends of body, the bottom can have one or more grilles or integrated louvers (openings) for movement of air and one or more UV modules (second source). In any of the above embodiments, the grilles or integrated louvers and the UV module can be placed on the bottom of the body at the ends, after where the door ends. In any of the above embodiments, one UV module can be placed at each corner of the body. In any of the above embodiments, the ends of the body can have intake/exhaust for the air, which is forced inside the body with one or more fans. In any of the above embodiments, the fixture comprises an airway/conduit for passage of air from outside, such as through openings/grilles/louvers and/or filters 20, to the first irradiation source, and passage of the irradiated and/or filtered air to outside of the body. In any of the above embodiments, the light from the UV light inside of the body (first source) is retained inside of the body. In any of the above embodiments, the UV light inside of the body can be placed behand a removable panel that is above the door. The panel has to be opened to access the UV light chamber. In any of the above embodiments, the chamber can have an air baffle on top preventing UV light rays from escaping through the grilles or integrated louvers and creating turbulent flow in the irradiation chamber. In any of the above embodiments, the chamber can have an air riser scoop that directs air into the irradiation chamber and reduces reflection. In any of the above embodiments, one or more light sources to provide illumination can be placed below the door. In any of the above embodiments, the light sources, such as LED or OLED, can be coupled with a suitable diffuser/lens, and/or reflective back plate/paper.

In any of the above embodiments, the disinfecting agent can be a wall mounted fixture, comprising a) a body; b) fan; c) a first irradiation source inside of the body, the irradiation source inside of the body configured to disinfect air forced into the body by the fan; d) an optional second irradiation source or sources configured to irradiate outside of the body; e) optionally one or more light sources to provide illumination. In the wall mounted embodiment, the fixture that is mounted on the wall can have a vertical orientation, with horizontal grilles or integrated louvers on the top and/or bottom of the front face of the fixture, a mounting mechanism on the back or on the sides, and an optional lighting source, such as in a horizontal orientation. The wall mounting unit can lack a pivoting door. The wall mounting unit can have access to the UV chamber inside the unit from the front face of the unit. The wall mounting unit can have any of the embodiments discussed above, including one or more UV lights on the outside, irradiation chamber, diffuser light, UV irradiation modules, air intake grille, exhaust grille, motion sensor, and other optional sensors and electronic components.

### DETAILED DESCRIPTION OF THE INVENTION

FIG. 1 shows a bottom view of an embodiment with the light panel open, for example for maintenance or lamp or filter replacement. Illustrated in FIG. 1 are UV (Ultra Violet) irradiation chamber 1, body 2, UV irradiation chamber access door 3, main access door 4, optional downlight 5 (can also be a decorative element without a light source), UV irradiation modules 6 with UV light source 77, air intake grille 7, and exhaust grille 8.

The disinfecting system can have two different embodiments. In one embodiment, the disinfecting system is configured only to disinfect/purify the air in a room. In another embodiment, the system is configured in addition to disinfecting/purifying, to also illuminate a room by having one or more light sources. The one or more light sources can be LED (Light Emitting Diode) or OLED (Organic Light Emitting Diode) light sources 61.

The disinfecting system can be in form of a fixture with a body 2 that is configured to be attached to a ceiling. The fixture can have a main access door 4 that is pivotally attached to one side of the body 2, allowing a technician to open the door 4 from the bottom of the fixture and access the UV light source(s) and filter(s) inside of the body 2. In the version with light sources providing a down light, the light sources can be placed directly on a bottom side of the main access door 4. In the version of the fixture with no downlight, the main access door 4 can be plain or have a decorative panel.

The body 2 of the fixture can have one or more grilles or integrated louvers (7, 8), and/or one more irradiation modules 6 that are placed anywhere around the perimeter of the access door 4. The bottom surface of the grilles or integrated louvers (7, 8), and/or one more irradiation modules 6 can be flush with the bottom surface of the body 2 or recessed from the bottom surface of the body 2. In one embodiment, the grilles, or integrated louvers_(7, 8) are placed at the ends of the body 2 in the center, with the irradiation sources placed at one more or more, or all of the corners of the body 2. Alternatively, the irradiation sources can be placed along the long edge(s) of the body 2.

Under the grilles or integrated louvers (7, 8) are one or more fans 19 and a filter 20. The fans 19 bring the air into the unit and into the irradiation chamber. The filter 20 functions to remove large dust particles and to keep the irradiation chamber clean. The exhaust grille 8 directs the clean air out of the unit to circulate within the room. There may be a separate filter on the exhaust grille 8 to catch deactivated pathogens.

The UV modules may contain LEDs, low pressure Mercury lamps, or far UV lamps, which are in the 207 nm or 222 nm range. These lamps can be Krypton-Bromine or Krypton-Chlorine Excimer Lamps. The UV Irradiation modules 6, in the embodiment are placed on each corner of the system (such as from 0 to 5 inches from the edge of any inlet or exhaust point), and installed on units primarily to provide air and surface disinfection when the room is not occupied. They may use UV light, with wavelengths anywhere from 200 nm to 280 nm. The modules may alternatively be provided as two tubular or longer light sources. Alternatively, one or more longer UV light sources may be placed along the edges of the unit. The surface disinfection modules combine with the air purification system to reduce viable pathogens in the area below the system. In another embodiment, UV light having wavelengths of 207 nm or 222 nm may be used, for example, so that the surface infection modules can remain on when the room is occupied. The UV modules allow the system to more effectively disinfect the air in the room before the air has been drawn into the irradiation chamber intake, and disinfect surfaces below the fixture. In another embodiment, UV light of 253.7 nm may be used to provide both surface disinfection and air disinfection. In other embodiments, the modules can operate either continuously or intermittently, based on information from the sensors or from a scheduled cycle. For example, the modules may be programmed to operate when the motion sensors 50 detect that someone has entered the room, or, alternatively, when they detect that someone has entered and left a room. In an alternative embodiment, sound sensors 51 may be provided to detect human activity in the room, and the system is programmed to operate the modules in response to detected sounds. In one example, the system may detect the sound of a person sneezing or coughing, and in response, activate the modules for a period of time to disinfect the air.

A motion sensor 50 can be used if harmful UV rays are used in the irradiation modules to ensure that they are not on when anyone is in the room.

Other sensors can also be used. Another sensor, such as a heat (temperature) sensor 52, may be used if the unit is installed in patient rooms or ICU (Intensive Care Unit) rooms or similar areas where there are patients who do not move so that the surface disinfection modules do not function.

The fixture can have other electrical components, including, but not limited to, ballast/controller 53, power supplies (power management unit 57), a UV light measurement sensor 78, a safety switch for the UV access door, wiring, and controls for remote operation 58 and indicator 56. The control system can use a system (which may be wireless 54) that will be used to operate the functions of the unit (UV air system, UV surface system, down light on/off, down light dimming) where they can be manually turned on or off (various switches 55), or programmed to be on a schedule. Indicator 56 can also indicate any notifications or errors such as: Filter replacement, UV lamp replacement, fan malfunction, UV module replacement, UV intensity in the irradiation chamber, unit not able to come on as scheduled due to motion sensor activation, unit not able to come on as scheduled due to other (heat) sensor activation, UV chamber door open, and downlight malfunction.

FIG. 2 illustrates the top section of irradiation the chamber. Illustrated in this drawing are UV irradiation chamber body 9, UV irradiation chamber reflector 10, air baffle top 11, air riser scoop 12, and UV light source(s) 13. The UV irradiation chamber reflector 10 increases UV exposure to the air as it passes through the unit. The air baffle (top) 11 prevents UV light rays from escaping through the grilles or integrated louvers and creates turbulent flow in the irradiation chamber. The air baffle 11 can be curved. The air riser scoop 12 directs air into the irradiation chamber and reduces reflection. In FIG. 2, the UV light source 13 is shown as a low-pressure mercury lamp, but could also be any UV light source such as LEDs, or Kr-Cl sources.

FIG. 3 illustrates the body 2 assembly. Illustrated in this figure are main troffer body 14, front panel for troffer body 15, access door for UV irradiation chamber 16, air baffle bottom 17, and fan tray 18. The fans 19, and filter 20 that are placed in fan tray 18 are illustrated in detail in FIG. 10.

The front panel for troffer body 15 can include louvres for the intake and outlet vents. The access door for UV irradiation chamber 16 allows accessing the UV lamp for replacement. There can be a momentary disconnect switch 55 that will turn off UV lamp if the access door is opened. The air baffle bottom 17 can prevent UV light rays from escaping through the grilles or integrated louvers and creates turbulent flow in the irradiation chamber. The fan tray 18 can mount fans 19 in place and holds filter 20 in place. FIG. 10 illustrates an exploded view of the fan including the fan 19 and filter 20. There can be 1-6 fans 19 at one or both ends, such as 4 fans are used in this version. The filter 20 can be a MERV6 filter for removal of large dust particles in order to keep the irradiation chamber clean. There can be one or two fan trays 18. Typically one fan tray 18 is placed where the air enters the fixture.

FIG. 4 illustrates a UV LED board placed along the length of the body 2. The LED board 26 can also be present only for the purposes of illumination, not irradiation. In this embodiment, the front access door 4 can be smaller in width to accommodate for placement of the LED board 26 containing LED light sources.

FIGS. 5 and 6 illustrate a wall mount version of the disinfecting system. The body 70 as illustrated is configured to be mounted on a wall or recessed into a wall in a vertical fashion, with grilles or integrated louvers 71 on both the top and the bottom of the body 70. An optional light (for illumination) 72, placed such as in a horizontal manner, can run parallel and below the front facing grilles or integrated louvers 71 on top of the body. The wall mounted version can have a flat front and a panel 73 that is accessible from the front of the unit. A pivoting door can be absent altogether from front of the wall mounted unit. FIG. 6 illustrates placement of LED boards 60 and modules 69, which can be placed on the front face of the body. The unit can also have UV boards and modules placed on the front face of the body 70 or panel 73.

FIG. 7 illustrates various electronic components of the disinfecting system. Illustrated in this figure is motion sensor 50, which can sense whether an individual is or is not present in a room, and accordingly switch 55 may turn on or off external UV lights that are not safe for individuals. Sound sensor 51 can also be used to sense the presence of people, and can be used alone or in conjunction with motion sensor 50. The fixture can have a temperature sensor 52 for determining heat sources within the room. The fixture can have ballasts/controllers 53 and/or a power management unit 57, for managing power to the UV and white lights, sensors, and fans. The fixture can have a wireless controller 54, such as with Bluetooth or Wi-Fi, to allow a user to turn on and off the fixture, and/or control the different features of the fixture. The fixture can have switches 55, which can be turned on and off manually, with the wireless controller 54, or internally if a panel is opened by a technician or the fixture needs maintenance. The fixture can have one or more indicators 56, which can indicate when the UV light is on in the chamber and/or outside of the chamber, and/or indicate the need for maintenance, such as replacement of the UV light. The wireless Controller can send information on required maintenance or system performance to an outside monitor and control system such as a computer, tablet, or smart phone.

FIG. 8 illustrates an LED board 60 with LED sources of light 61. The LEDs can be placed in one or more rows over the board. The LEDs can be one or different colors, and some instances one set of LEDs producing only UV light while other producing while light for purposes of illumination.

FIG. 9 illustrates placement of various UV and/or non-UV lights outside of the body. One or more of the light sources illustrated in FIG. 9 can be used, such as light 72, 74, 75, and 76. Visible in this figure is the transparent diffuser 75 for the light source, which is placed in the Main Access Door. The lights can be used in pairs. The lights can be dedicated UV lights for the purpose of disinfection or provide light in the visible spectrum, including white light for the purpose of illumination, and/or provide both visible light and UV disinfecting light.

FIG. 10 illustrates exploded view of an assembly with a fan 19 and filter 20. Illustrated in this view are four fans 19, which are placed in fan enclosure 25. Fan Tray 18 holds the fan enclosure 25 and filter 20. The assembly can have a service panel 24 configured to allow for change of the filter 20 and/or fans 19.

FIG. 11 illustrates a top view of the fixture. Illustrated in FIG. 11 are the UV irradiation chamber 1 and body 2. Illustrated in this view are ballast and controllers 53. There can be two or more ballasts and controllers 53. One set of ballasts and controllers can control the light, another set the and sensors, and another for the UV lamp. Ballasts and controllers can control the UV modules or lamps for irradiating air and surfaces outside the fixture. The fixture can also have a UV sensor 78 which measures the intensity of the UV light in the irradiation chamber and can output the reading to monitor the effective intensity.

Also provided are methods for irradiating air, and disinfecting air with a fixture. The method includes using any fixture as described above to disinfect the air. The method can include sucking air inside of the body of the fixture with one or more fans 19, irradiating the air inside of the fixture, and pushing the irradiated air out of the fixture with the same or different fans. The method further includes irradiating air outside of the body with an additional irradiation source. The method can further include irradiating air outside first with an outside irradiation source, and then with an inside irradiation source. The method can further include irradiating air inside of the fixture first with an inside irradiation source, and then outside of the fixture with an outside irradiation source. The method can further include irradiating air outside of the fixture, then inside of the fixture, and then outside of the fixture.

### REFERENCES

1. UV Irradiation Chamber
2. Body
3. UV Irradiation Chamber Access Door
4. Main Access Door
5. Downlight
6. UV Irradiation modules
7. Air intake grille or louver (Openings)
8. Exhaust grille or louver
9. UV Irradiation Chamber Body
10. UV Irradiation Chamber Reflector
11. Air Baffle Top
12. Air Riser Scoop
13. UV Light Source
14. Main Troffer Body
15. Front Panel for Troffer Body
16. Access Door for UV Irradiation Chamber
17. Air Baffle Bottom
18. Fan Tray
19. Fans
20. Filter
21. Door/Reflector Body
22. Reflector
23. Diffuser
24. Service Panel
25. Fan Enclosure
26. LED Board
50. Motion Sensor
51. Sound Sensor
52. Temperature Sensor
53. Ballasts/Controllers
54. Wireless Controller
55. Switch
56. Indicator
57. Power Management Unit (PMU)
58. Wiring and Controls For Remote Operation
59. N/A
60. LED board
61. LED source of light
69. UV-C Module
70. Body/Body
71. Grilles
72. Optional Light
73. Panel
74. Light
75. Light
76. Light
77. UV light source
78. UV Sensor

What is claimed is:

1. A disinfecting fixture comprising:
a) a body;
b) a fan;
c) a first irradiation source placed inside of the body, the irradiation source inside of the body configured to disinfect air forced into the body by the fan;
d) a conduit for movement of air from outside of the body to inside of the body for irradiation with the first irradiation source, and movement of air outside of the body after irradiation with the first irradiation source; and
e) a second irradiation configured to irradiate outside of the body;
wherein air moves with a force generated by the fan from outside of the fixture to inside of the fixture for irradiation with the first irradiation source, and exits the fixture, and the second irradiation source irradiates outside of the fixture.

2. The disinfecting fixture of claim 1, further comprising a door attached to the body for accessing inside of the body.

3. The disinfecting fixture of claim 1, further comprising one or more light sources placed outside of the body to provide illumination with at least a non-UV light wavelength.

4. The disinfecting fixture of claim 1, further comprising a door attached to the body, the door comprising one or more sources of light for illumination outside of the fixture.

5. The disinfecting fixture of claim 1, further comprising one or more openings placed on the body for movement of air.

6. The disinfecting fixture of claim 1, wherein an edge of the second irradiation source is placed zero to five inches to nearest location where air enters the conduit from outside.

7. The disinfecting fixture of claim 1, wherein the second irradiation source irradiates a portion of the air entering the fixture.

8. The disinfecting fixture of claim 1, wherein the second irradiation source irradiates a portion of the air leaving the fixture.

9. The disinfecting fixture of claim 1, wherein the second irradiation source is in form of a module.

10. The disinfecting fixture of claim 1, wherein the second irradiation source is placed in on or more corners of the fixture.

11. The disinfecting fixture of claim 1, wherein radiation from the first irradiation source is retained inside of the body.

12. The disinfecting fixture of claim 1, wherein the first irradiation source is placed in a chamber behind a removable panel.

13. The disinfecting fixture of claim 12, wherein the chamber had an air baffle that prevents UV light rays from escaping the body.

14. The disinfecting fixture of claim 12, wherein the air baffle is curved.

15. The disinfecting fixture of claim 12, wherein the chamber has an air riser scoop that directs air into the irradiation chamber.

16. The disinfecting fixture of claim 1, wherein the fixture is configured to be mounted on a wall.

17. The disinfecting fixture of claim 1, wherein the fixture is configured to be mounted to the ceiling.

18. The disinfecting fixture of claim 1, wherein the second irradiation source is 0 to 5 inches from the edge of any inlet or exhaust point where air enters to leaves the fixture.

19. The disinfecting fixture of claim 1, further comprising one or more light sources placed outside of the body, the light sources producing differently colored lights.

20. A method for disinfecting air or surfaces comprising:
a) creating a flow from outside to inside and then outside of a fixture with a fan;
b) irradiating the air moved inside of body;
c) moving the irradiated air outside with the flow of the air; and
d) irradiating air or surfaces outside of the fixture with a second irradiation source placed outside of the body.
